(19) United States
(12) Patent Application Publication
(10) Pub. No.: US 2020/0135305 A1
(43) Pub. Date: Apr. 30, 2020

(54) PROCEDURE FOR UNIFIED GLOBAL REGISTRY AND UNIVERSAL IDENTIFICATION OF PRODUCTS OF BIOLOGICAL ORIGIN FOR MEDICINAL PURPOSES

(71) Applicant: CONÉCTATE SOLUCIONES Y APLICACIONES SL, Soria (ES)

(72) Inventors: Fernando Latorre López, Soria (ES); Nuria Sala Cano, Soria (ES)

(21) Appl. No.: 16/169,126

(22) Filed: Oct. 24, 2018

(51) Int. Cl. G16H 10/40; G16H 15/00; G16H 80/00

(52) U.S. Cl. CPC G16H 10/40; G16H 80/00; G16H 15/00

(57) ABSTRACT

A procedure for the unified global registry and universal identification of products of biological origin for medicinal purposes comprises:
Generation of a global and unique data storage vault in the system for each product and related parties, one or more local data vaults associated with the global storage vault, one or more data profiles associated with the vaults, a unique and non-transferable public and private identifier associated with the global storage vault, one or more alphanumeric-hexadecimal translation maps associated with the public and private identifier, and a unique and non-transferable public identification code, and store these data in a database accessible by different parties depending on the data access rights.
Identification of the data storage vault and the type of data stored through the unique and non-transferable identification code and automatic updating the information in the vaults and profiles stored in the database accessible by different parties.

### TECHNICAL FIELD

The present invention is generally encompassed in the health sector, donations, manufacture of medicines of biological origin, tissue engineering and data management, falling particularly in the field of: centres for the obtention or collection of donations of blood, plasma, cells, tissues and organs; centres or laboratories for the generation of tissues and organs; centres destined to carry out transplants and transfusions; pharmaceutical companies producing blood products; laboratories for the production of medicines of biological origin; and data management systems. More particularly the invention relates to a procedure for the unified global registry and universal identification of products of biological origin for medicinal purposes in humans or in other living beings and of the different related parties.

### BACKGROUND OF THE INVENTION

Obtaining and using materials and products of biological origin involves certain specific considerations arising from the nature of the products and the processes. The ways in which biological products are obtained, manufactured, controlled and administered make some particular precautions necessary. The obtention or manufacture of products of biologic origin may involve processes such as cultivation of cells or the extraction of products from living organisms and carry a risk of transmission of diseases. While this risk is minimized through testing and processing, it can never be completely eliminated (e.g. the detection of a serious infectious disease in a donor after having made one or more donations of one or more types over time).

Worldwide, 112 million donations of blood and plasma of human origin are collected annually and more than 120,000 human organ transplants are performed, although the WHO recognizes that this FIGURE is only 10% of the transplants that are necessary each year. In addition, the average life of a transplanted organ is 10 years if it comes from a deceased human donor and from 14 years if it comes from a living donor.

Some of the products of biological origin for medical purposes, such as blood and its derivatives or organs, have a very short shelf life and have to be kept under very strict temperature conditions to ensure their quality and that they do not suffer deterioration. For example, each blood donation is fractionated to separate its components (red blood cells, plasma and platelets mainly) and make blood products (products derived from blood). A unit of red blood cells (RBC) is obtained from 3 blood donations and an expiration of up to 42 days is achieved using preservatives such as Sagmanitol, provided that it remains between 1 and 6 Celsius degrees, although there are studies that claim that Red blood cell deterioration occurs as the time since the blood was collected increases. In the case of platelets, the expiration is from 5 to 7 days. The plasma can be maintained for up to 2 years at a temperature of 30 degrees centigrade below zero. Tissues and organs such as the heart or lung have an average preservation time of between 3 and 5 hours after extraction, while skin and bones can be preserved for up to 5 years.

Some blood products, such as units of red blood cell (RBC) or platelets (PLT), are usually not sent to other countries except in some emergency cases. However blood plasma is exported from the U.S. to the rest of the world to manufacture drugs such as vaccines or hemophilia medications from immunoglobulins. In fact, approximately 65% of the global plasma comes from the U.S. and nearly half of the U.S. plasma is exported to Europe.

A donor can be the source of many different products. For example, a deceased donor can donate skin, tendons, heart valves and a wide range of bone products. All these different products share a common history. Some of the products obtained from a donor may fall within the regulation of biological products, while other products from the same donor may correspond to the regulation of medical devices or even not be considered medicines (e.g. creams).

Biological products are products of biological origin. They include a wide range of products such as vaccines, blood and blood components, allergenics, somatic cells, gene therapy, tissues, and recombinant therapeutic proteins. Biological products can be composed of sugars, proteins, or nucleic acids or complex combinations of these substances, or may be living entities such as cells (ovules, red blood cells, . . . ), tissues and organs. They are isolated from a variety of natural sources (human, animal, vegetable, microorganism) and may be produced by biotechnology methods and others technologies.

For biological medicinal products, starting materials shall mean any substance of biological origin such as micro-organisms, organs and tissues of either plant or animal origin, cells or fluids (including blood or plasma) of human or animal origin, and biotechnological cell constructs (cell substrates, whether they are recombinant or not, including primary cells). A biological medicinal product is a product, the active substance of which is a biological substance. A biological substance is a substance that is produced by or extracted from a biological source and that needs for its characterization and the determination of its quality a combination of physico-chemical-biological testing, together with the production process and its control.

The manufacture of biological medicinal products is a highly demanding process. Protein-based therapies have structures that are far larger, more complex, and more variable than the structure of drugs based on chemical compounds. Plus, protein-based drugs are made using intricate living systems that require very precise conditions in order to make consistent products. The manufacturing process consists on producing the master cell line containing the gene that makes the desired protein, growing large numbers of cells that produce the protein, isolating and purifying the protein and preparing the biologic for use by patients. Some biologics can be made using common bacteria, others require cell lines taken from mammals. Each biotechnological process starts with the creation of a unique cell line to develop the desired medicine and at each stage of this process, the biological medicine acquires a series of properties that make it different from the rest. Therefore, due to the great diversity of factors that can vary in the process of obtaining these drugs, the final activity in vivo can be altered, either by experiencing reductions in the therapeutic effect or by the appearance of immunogenicity.

Biological drugs have some potential to be recognized by the body as foreign substances and, consequently, have the inherent ability to induce unwanted immune reactions due to their composition and their high molecular weight. One of the most important issues in the appearance of immunogenicity reactions is the possible variation in the glycosylation pattern experienced by this type of drugs, which can lead to unpredictable clinical consequences. This potential to induce immunogenicity on the part of biological medicines is why pharmacovigilance and the traceability of these medicines acquire special importance, with the aim of ensuring and promoting their safe use.

Biosimilar medicines are defined as biological products “similar” to other biotechnological medicines already authorized, produced by a different manufacturer of the biological reference drug, using different cell lines, different processes and analytical methods. Although the biological substance of a biosimilar and its reference medicine is essentially the same, there may be certain differences related to its complex nature and method of elaboration. Due to the complex process, hundreds of irreproducible phases to which the different cell lines are subjected, it is impossible to guarantee the production of completely identical biological products.

The inclusion of batch and lot records allows the rapid detection of any problem related to specific lots or batches of a product and allows the grouping of medicines by shared attributes (such as active pharmaceutical ingredient), brand name or manufacturer, specific lots and batches of a sole medicinal product. The maximum combination of data can lead to more timely decisions in pharmacovigilance.

There are different labeling systems for products such as ISBT, HIBC or GS1. Currently the products of biological origin obtained in a certain centre carry a product identification code generated by one of these systems. In addition, different unified codes for medical devices (UDI) have been implemented currently in some countries and are being developed in others to achieve a better traceability of the products in each territory. However, these UDIs are independent and different of each other and their databases are intended to be isolated (Europe, USA, Australia, . . . ). On the other hand, and apart from the UDI, other products of biological origin have to be identified with other specific codes such as the Single European Code (SEC) for tissues and cells in Europe.

In addition to the above, the traceability of the origin of biological materials or products are limited at the level of a specific organization or territory, due to the isolation of existing data between different systems, organizations and territories, as in the case of donors or recipients. Until now, the identifier of a blood or organ donor was at the local level for a public or private organization or for a specific territory. The same happens with an individual recipient of a tissue or organ transplant, of a transfusion of blood products such as red blood cells or of a vaccine in which blood plasma has been used in the manufacture, since the identification and traceability of the individual is currently a local code of user, client or patient for a specific public or private medical or hospital organization or a health code for a territory, whose history is isolated from other centres or territories.

The application US-2018-0247346-A1, which in turn is a continuation of the patent PCT/ES2016/070428 and which in turn is a continuation of the patent in Spain P201531611, now allows a unified global registry of blood donors and other types to be available. This makes knowledge of the history of a donor in any donation centre possible and thus improve the traceability and safety of suppliers of these biological products in different public or private organizations and in different territories. This also allows knowledge of other relevant health data related with the donor, which makes it possible to improve the safety of the products (frozen plasma unit, tissues, organs, . . . ) and a universal traceability from donor to recipient, together with the possibility of minimizing the illegal black market of certain products.

However, there is no known global system that allows the monitoring of the recipients of products of biological origin (transfusion, transplant, vaccination, . . . ) beyond the organization or specific territory where the process is carried out, and that can allow a universal traceability from recipient to donor in an automatic way. There is also no known global system to register the use of products of biological origin in a unified record of clinical interventions or treatments of this type and that can allow traceability of centres, products used and recipients thereof (users, clients or patients). For example, in a clinical intervention to perform a tissue implant, transfusions of blood products may be required, but there is no known unified system that allows a global automatic post-traceability of the products of biological origin used in a specific recipient, client, user or patient, as in the event that some kind of adverse effect occurs in the short, medium or long term and the recipient is treated in a medical centre of another organization and/or different territory. There is also no known unified global system to automatically alert to this individual and/or to the medical reference centre of an anomaly detected in one of these products to take the appropriate actions.

On other hand, labeling products with printed labels has some drawbacks. For example, the label may be misplaced in a similar product or may deteriorate and hinder product identification. This is very important for example when labeling blood bags, as each blood bag has to be associated with a specific donor with a specific blood group and is also associated with a blood sample tube with which blood tests are performed to find out if the donated blood is valid or has to be discarded according to the blood test result.

Nevertheless every NFC chip has a globally unique, manufacturer supplied, read-only identifier (UID) that can be read by most NFC devices. An NFC tag's UID cannot be changed or erased; it is stored in special memory in the NFC chip which does not allow the bits to be changed. So this technology can be used to identify a product worldwide, such as a donated pint of blood, and also can be associated with the internal common identifier given for the donation centres to commonly identify donors when using the unified registry of donors.

The classification of medical devices bind manufacturers to determine the product classification in order to comply the Medical Device Directives, For example, blood, human cells, tissues or organs intended for implantation, transplantation, infusion, or transfer into a human recipient are regulated as medical devices in some countries. But the Medical Device Directives are different in each country.

A Medical Device, as is described by the International Medical Device Regulators Forum (IMRF), means any instrument, apparatus, implement, machine, appliance, implant, reagent for in vitro use, software, material or other similar or related article, intended by the manufacturer to be used, alone or in combination, for human beings, for one or more specific medical purpose(s). Some products may be considered to be medical devices in some jurisdictions but not in others, as devices incorporating animal and/or human tissues, for example.

In Europe, as in the above reference, the definition of Medical Device only include humans as recipients, not animals. But the Food and Drug Administration (FDA) define medical device for pre-marketing and post-marketing regulatory controls in this way: “an instrument, apparatus, implement, machine, contrivance, implant, in vitro reagent, or other similar or related article, including a component part, or accessory which is:
recognized in the official National Formulary, or the United States Pharmacopoeia, or any supplement to them,
intended for use in the diagnosis of disease or other conditions, or in the cure, mitigation, treatment, or prevention of disease, in man or other animals, or
intended to affect the structure or any function of the body of man or other animals, and which does not achieve its primary intended purposes through chemical action within or on the body of man or other animals and
which does not achieve its primary intended purposes through chemical action within or on the body of man or other animals and which is not dependent upon being metabolized for the achievement of its primary intended purposes. The term “device” does not include software functions excluded pursuant to section 520(o).

So animals are included as recipients by the FDA, not by the IMRF nor the E.U. Also, distinctions are made between a medical device and other regulated products such as drugs. If the primary intended use of the product is achieved through chemical action or by being metabolized by the body, the product is usually a drug. For example, human drugs are regulated by the FDA's Center for Drug Evaluation and Research (CDER), but biological products which include blood and blood products are regulated by FDA's Center for Biologics Evaluation and Research (CBER). And, finally, the FDA's Center for Veterinary Medicine (CVM) regulates products used with animals.

The nomenclature of medical devices is a coding system used to describe medical device categories and to generically identify medical devices and related health products. Product classification and nomenclature in the global healthcare sector is quite complex. Several naming systems for medical devices exist and each is used by a different group of professionals depending on the needs of that particular group, needs such as maintenance, procurement, accounting, stock keeping, regulatory affairs, adverse medical device event reporting, and customs operations (CMDR, DM&D, eCl@ss, GMDN, GPC, HCPCS, MedDRA, NAPCS, NHS eCl@ss, UMDNS, . . . ). There are also on the market different types of labeling systems for devices and products for medical use (e.g. GS1 or HIBC). On other hand, ISBT 128 is intended only for products of human origin.

Supply chain management and optimization has become a priority area for global health systems due to increasing complexity of healthcare supply chains and the availability of IT solutions. Globally, supply chain transformation is becoming a priority area for many healthcare systems who are aiming to control rising costs

A UDI is a unique numeric or alphanumeric code, usually in the form of a barcode, that can be displayed in both human readable (plain text) and machine readable (AIDC) form; it identifies a device at the point of distribution and at the point-of-use that consists of two parts: a device identifier (DI) and production identifier(s) (PI). The Device identifier (DI) is a mandatory, fixed portion of a UDI that identifies the specific version or model of a device and the labeler of that device. The production identifier (PI) is a dynamic number that is determined by various data (such as batch number, serial number, expiration date, and date of manufacture).

There are different initiatives throughout the world to develop Unique Device Identifiers (UDI) in each country or region; different countries have developed or are developing their own databases and identification systems for medical devices, such as the FDA GUDID for U.S. and EU-UDI for Europe, for example. These systems are independent of each other and, therefore, keep the data isolated in different databases. They also differ in the fields that define each product and in the way of identifying the labeled products.

In Europe, for example, UDI carrier contains DI+PI in a machine (AICD) and human (HRI) readable representation. In U.S. only AICD is needed. AIDC data carrier is based on ISO standards (bar code, data matrix, RFID) and the data is formatted in a code standard (e.g. GS1, HIBCC). In Europe the AIDC format is favoured in case of significant space constraints on the device or packaging; however, the use outside of healthcare facilities requires HRI on the label.

European regulations requirements on generating clinical evidence on medical devices across their entire lifecycle. Europe MDR/IVDR requirements for some devices is “to continuously generate, collect, analyze and assess the clinical data pertaining to a device in order to verify the safety and performance, including the clinical benefits, of the device when used as intended by the manufacturer.”

Globally harmonization goes beyond making it easier for manufacturers who have to comply with regulations in many different countries. Using a common approach to product identification provides important downstream benefits for regulators, providers and patients as well. Given that many devices sold around the world are similar if not the same across various countries, the data generated on a device in one part of the world can be relevant for data consumers elsewhere.

Inconsistency in the format used to identify medical devices and the lack of a harmonized nomenclature and structure for medical device identification information, currently result in an inability to compile effective market surveillance information about a medical device or product. Lack of a common unified identifier for a universal medical product identification also makes it possible that a medical device may be referenced distinctively in different countries, which limits the ability to compile data or make comparisons across countries. The invention that is intended to be patented allows for cooperation between the different stakeholders (individuals, organizations, regulators, distributors, etc.) in order to improve the traceability of the products worldwide, create alerts, carry out studies and follow-up on donations, blood transfusions, tissues and organ transplants and other related medicines.

At present there is no known the existence of a system that allows a unified record and a universal identification of biological products of human or animal origin for medical ends in a cross-border way between different countries and systems of codification of products, that could be used from any place in the world to register the deals carried out on these products (like collection/delivery, purchase/sale, change of owner, . . . ), and to be aware of alterations in the conditions of the product along the whole life of the same one from its origin or production up to its use, which could bear the automatic rejection of the product and prevent it or its derivates from being used in case of some anomaly being detected.

Consistent use of a universal identifier for biological products from human or animals and for medical or veterinary purposes will solve the above problems by providing a common way to refer to a product and enables tracking and reporting unambiguously on the regulatory marketing status of a medical device around the world, as control of biological medicines such as vaccines and blood products, and is essential for making sure the biological medicines that patients receive (humans or animals) are safe and effective. However, the existence of a unified and universal system for the global identification and traceability of products of biological origin is not known, nor is there a unified global register of clinical trials and of organizations that manufacture products of biological origin for medicinal purposes.

On the other hand, as has been mentioned above, there is no known unified global register of individual receivers or clients of biological products for medicinal purposes of any kind (transfusions, transplants, vaccines, etc.). Currently, if an individual receives a blood transfusion in a certain centre and a time later it is discovered that the origin donor of the product has suffered cancer and wants to perform a traceability of the recipients of the product or products, the isolation of data between the different centres prevents this in practice, unless the new centre belongs to the same organization as the other centre and uses the same data management system, perform an automatic and universal traceability of the individuals receiving products of biological origin for medical or veterinary purposes, and it is not possible to identify these recipients in any other centre than the one in which the product was used. For example, if an individual resides in a different region or country, currently that individual cannot relate to a universal identification code of a recipient or client of a biological product for medical or veterinary purposes, nor can this information be automatically related to a donation centre in case the person is a donor. This produces in practice that, for example, neither the recipient nor the medical staff from another centre in a different organization, region or country can receive an automatic alert to perform medical tests and follow-up after any anomaly is detected in a specific product of biological origin as well as automatically excluding the individual to donate during a certain time.

A complete cross-border traceability between different territories is necessary, labeling systems and regulations, including universal traceability from the origin of the biological products (obtention or manufacture), to the batch of the products, their use and vice versa, as well as being able to notify intermediaries that have used material of biological origin in a production process any event that may affect the quality or safety of the product and any other information found after obtaining the material of biological origin related. The principles of risk management for quality (Quality Risk Management, QRM) are, therefore, especially important. In addition, the data necessary for complete traceability must be retained for at least 30 years in some countries.

An example of use of what this invention allows would be the one described below. A regular blood donor dies and, after performing an autopsy, tumor cells or a tumor is detected in the deceased donor. Thanks to the existence of a unified global registry of products of biological origin for medical purposes and of the different related parties, this situation could be alerted automatically to all the related centres, as well as to quarantine all the products that have been manufactured from the previous blood donations from the donor and follow up on the products that have already been used (platelet units, blood units, plasma, manufactured vaccines, etc.) to take appropriate actions, such as monitoring of clients, patients and/or users who have received these products, allowing, on one hand, to study in a transversal and in depth manner these cases to be able to establish additional measures or protocols based on the results obtained, and on the other hand, to study the appearance or not of this type of disease in all the individuals involved.

In conclusion, and as a reference to the current state of the art, it should be noted that, although different regulations, types of user registration systems and registration and labeling of medical devices and products are known on the market, at least on the part of the applicant, the existence of any one with technical and constitutive characteristics similar to those presented by the invention that is asserted here is unknown.

### SUMMARY OF THE INVENTION

The invention, as expressed in the statement of this descriptive report, refers to a unified global registration procedure for all territories of products of biological origin for medicinal purposes in humans or other living beings (such as blood, its components and its derivatives such as plasma-derived medicinal products; other tissues such as nerves and blood vessels; other cells such as ovules and master cell lines; organs; others products of biological origin such as insulins, . . . ), of the distinctive related parties (individuals origin and/or destination of the products such as donors, recipients, clients, users or patients both humans or other types such as animals; other parties involved in the chain of obtaining, manufacturing, processing, storage, transportation and use of these products, clinical interventions, . . . ) and for the universal identification of both any product and any of the parties related to it (individuals and entities such as organizations, companies, donation and transfusion centres, laboratories, clinics, hospitals, personnel, . . . ), which presents advantages and characteristics, which will be described in detail later, which infer an improvement of the current state of technique.

Products of biological origin for medical purposes have in common that they come from living beings or use in their manufacture materials that come from living organisms, and include, among others, blood products (transfusions of red blood cells, platelets, . . . ), tissues and organs for transplants, medicines of biological origin such as vaccines, cells, advanced therapies such as tissue engineering and other biological products such as biotechnological medicines (erythropoietins, insulins, . . . ).

The object of the present invention falls, specifically, in a procedure whose purpose is to achieve the unification of the registration and identification of the products of biological origin (obtained from donors or from other sources) and of the different parties related to the obtention and use of the these (entities, human or other individuals, . . . ) in order to facilitate the access and monitoring of the data in the entire chain of collection, production, supply and utilization of the products of biological origin (obtained from donations or from other sources) for medicinal purposes in humans or other living beings, in a universal manner, from the origin (human or of another living being) until the final use of the product in humans or in other living beings, to certify anywhere, and in a universal manner, the origin, the destination or recipient, as well as the intermediaries and the quality control of the products, in order to:
Facilitate, on one hand, the exchange of information related to the products (centre of origin or manufacturing, storage, distribution, receiving centre, clinical intervention, . . . ) and, on the other hand, the unification of the health data of the individuals that are the origin or the destination of the biological products for medicinal purposes (medical or veterinary);
Improve and facilitate the identification of individuals in a universal manner such as donors, recipients, users, clients and patients, humans or other types;
Facilitate, in a universal manner, the identification and access to the data of the products along the chain of collection, production, distribution, supply, utilization, etc., in a unified and universal way, from their origin (human or other types such as animals) until their final use in humans or other living beings;
Allow transverse traceability of products of biological origin between different territories, related parties and systems;
Record the transactions made with the product throughout the production, supply and utilization chain by all the parties involved;
Certify in any place and universally both the origin of the product and all intermediaries related to it;
Certify, control and track the quality of the products;
Save financial resources;
Increase the safety of donations, transfusions and transplants of any kind as well as other types of products of biological origin.

This procedure consists of setting up, by centres responsible for the obtention, generation or manufacture of the products (donation centres, hospitals, laboratories . . . ) and/or by the centres responsible for the use of the products (hospitals, clinics, . . . ) the following, by means of an application in an electronic device (computer, mobile, tablet, . . . ) and a computer system:
a global and unique data storage vault for each of the parties, as an individual (human or other types), product (such as units of plasma or red blood cells, immunoglobulins, vaccines, organs or tissues), or other parties (collection centre, manufacturer, clinical trial, clinical intervention . . . );
one or more vaults of local data associated with the global storage vault containing data stored in each geographical region or specific territory for compliance with different regulations (identification of products, storage of personal data, . . . );
one or more data profiles associated with each vault (global or local) which contain public information (such as blood group in the case of an individual and expiration date in the case of a product) or private data for use by the individual and/or the parties related to the product and authorized to access said private information, such as relevant information on the product (temperature control, tests performed, . . . ) or on the individual (diseases or relevant health disorders, clinical interventions, relevant medical or health treatments, prescription or recent consumption of medication, allergies, relevant medical reports, analytical tests, . . . );
an internal identifier to identify univocally in the system each generated profile and local vault, internally relating the information stored in the different profiles and local vaults with the unique global storage vault generated for a determined individual or product;
a unique and non-transferable public identifier associated with the global storage vault to uniquely identify the parties (individual, product, centre, . . . ) and access public stored data;
a unique and non-transferable private identifier associated with the global storage vault to identify the type of data it refers to (individual, product, collection or manufacturing centre, destination centre . . . ) for access by each related party to the data to which it has access, allowing anonymization and privacy of personal or medical data related to the individual and of private data related to the product;
a non-transferable public identification code that contains at least one field for storing the public identifier and, optionally, other fields for storing the private identifier and/or other data such as the blood group of an individual or product, or production date, batch number and/or expiration of a product.

The local storage vaults and the associated profiles relate the information stored (for an individual, product or other related parties) in the computer system with the different systems of databases and existing identification systems (such as personal data and profiles with medical data in the case of an individual, or production and storage data in the case of a product).

The global data storage vault, the local storage vaults, the profiles generated and the public identifier are associated with a unique and non-transferable identification code that allows access to public data and, optionally, to other data by including the necessary fields in the identification code. These data are deposited and stored in the database of a computer system accessible by the different related parties (individuals, producers, intermediaries, . . . ) based on the access permissions in each case.

The identification code contains a set of data defined in fields that identify it as unique and that can also be stored in the system database and includes, at least, the public identifier and, optionally, other data such as blood group, prescribed medication and contacts for emergencies in the case of an individual.

### BRIEF DESCRIPTION OF THE DRAWINGS

To compliment the description that is being made and in order to insure a better understanding of the characteristics of the invention, the present descriptive report is accompanied, as an integral part thereof, by a plan in which, with an illustrative and non-limiting manner, the following is shown:

FIG. 1 shows, by means of a block diagram, the flow diagram of the operation of the code, according to the invention.

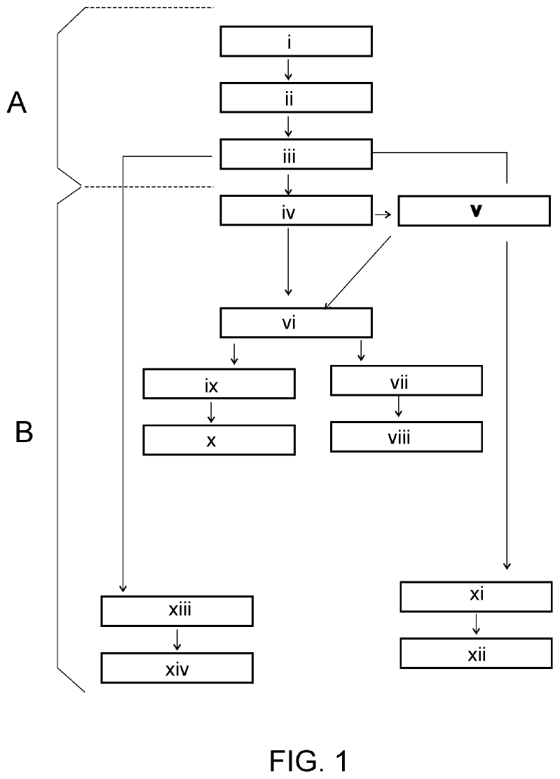

### DETAILED DESCRIPTION OF THE INVENTION

The procedure for the unified global registry and universal identification of products of biological origin for medicinal purposes in humans or other animals and of the different related parties proposed by the invention is configured as a novelty within its field of application, since according to its implementation the above-mentioned objectives are satisfactorily achieved, its characteristic details being conveniently collected in the final claims that accompany the present descriptive report.

As explained above, products of biological origin for medicinal purposes include, among others, blood products (transfusions of red blood cells, platelets, plasma, . . . ); medicines of biological origin such as vaccines; cells, tissues and organs for transplants; advanced therapies such as tissue engineering; and other products and biological medicines.

Specifically, what this invention proposes, is the development of a global process for the registration and for the universal identification of products of biological origin for medicinal purposes and of the different related parties such as individuals (donors, recipients, clients, users, patients), origins of products or biological materials (human, animal, cell lines, . . . ), entities related to the obtention, manufacturing, processing, supplying and utilization of products, clinical trials and clinical interventions or treatments in which products are used, whose purpose is to achieve the unification, in all territories and for any existing software solution, of the registration and identification system of these products and of the related parties, from the origin (human or another living being) to its final use in humans or other living beings, to facilitate the exchange of information of the products and between the different related parties as well as the traceability and cooperation between the different participants in the collection, production, storage, transport, processing, distribution and utilization phases.

The internal relationship between the authorized centres and the system of the invention will be carried out on a common computing medium for the exchange and storage of data, using one or more of these options, without excluding other possible embodiments: common API, VPN, blockchain, Smart contracts.

The unified global registry and the universal identifier that the invention proposes aim, on one hand, to identify the products, individuals and to each of the parties related to them in a global and univocal way and, on the other hand, to obtain and store the relevant information of all of them. In the case of products, it serves to identify them and to record the relevant events and transactions that take place with the products throughout the production and/or supply chain; in the case of individuals (such as donors, clients, users, patients), it serves to identify them in a global way and to follow up on their clinical history as well as to collect relevant medical or health information; in the case of other biological origins such as cell lines, it serves for identification (which may correspond to a lot number) and the recording of variations in production (growth conditions, compounds used to stabilize the protein, manufacturing conditions, . . . ); in the case of clinical interventions in which products of biological origin are used, it serves to relate the products, the centre that performs the clinical intervention and the individual that receives the product; in the case of collection, manufacturing, storage, supply and utilization entities, it serves to identify each one as well as authorized devices and personnel with connection to the system.

The procedure proposed by the invention is to unify and facilitate the monitoring of the whole chain of obtaining, producing and controlling the quality of the products, the supply and utilization chain, as well as retrieving information from these parts and the products, such as their origin, their destination, the manufacturer, the control of the storage conditions such as the cold chain, its utilization in a recipient, possible complications or adverse reactions, in order to facilitate and achieve a global monitoring of origin to recipient and of recipient to origin to achieve universal traceability in both directions.

The public identification code serves to identify a product or other related parties such as a collection or manufacturing centre, a donor, a cell line, a clinical, hospital or veterinary centre, a recipient, a user or a patient and it preferably has two types of information. A first type, of public information (open accessibility) such as the public identifier and other information such as blood group, date, place of origin, use of the product, expiration date and quality control, variable in each case. A second type of information will be accessible only from centres and personnel with the required permissions to decipher this information (e.g. health data of the individual, analytical data, production reports, etc.).

For this, said procedure essentially comprises of the following:
Creation and storage in a database accessible by the different centres and authorized users of
   a global and unique data storage vault for each of the parties, carried out after a voluntary request made by a public user (voluntary registration in the system) or by one of the related parties by means of an electronic device (computer, tablet, . . . ) and software with connection to the system;
   one or several local data storage vaults associated with the global storage vault containing data stored in each specific geographic region or territory;
   one or several data profiles for each of the related parties and associated with each data vault generated in the system, and that contains:
      public information of the product accessible to any user, and
      private information that is managed and shared by the different related parties;
   a unique and non-transferable public identifier in hexadecimal format associated with the global data storage vault to identify the type of data it refers to (individual, product, entity, clinical intervention, . . . ) and access public data stored in the data vaults and/or associated profiles;
   a unique and non-transferable private identifier in hexadecimal format associated with the global data storage vault to identify the type of data it refers to (individual, product, entity, clinical intervention, . . . ) and access the public and private stored data by authorized entities and personnel in each case;
   one or several alphanumeric-hexadecimal translation maps associated with the public identifier and the private identifier;
   a unique public identification code containing at least one field for storing the public identifier in alphanumeric format, generated from the hexadecimal public identifier and a concrete alphanumeric-hexadecimal translation map, and optionally other fields for storing the private identifier and/or other data;
Automatic update by the system of the data storage vault and/or profile data with relevant events and the date on which they occurred, related either to products, individuals or others (detection of a disease or prescription of a treatment in an individual, transactions or changes in the temperature conditions of a product, utilization of a product in a clinical intervention, . . . ).
Identification of the global data storage vault and the type of data stored (product, individual, clinical intervention, entity, . . . ) by the system executed by a public user or by an entity and its authorized personnel through the public identification code, for access to the data warehouse of the vault and to the stored local data vaults and profiles; generation, registration and communication of an incident if there is an impediment; and automatic updating of the data of the vault and of the profiles in the database accessible by the different parties and users.

Should be understood as relevant events those that may affect the safety of the product, any of the procedures or processes or the individuals involved, such as diseases, medications or treatments of an individual and variations in the temperature of conservation of a product, elapsed time without receiving information about the product, expiration of the product due to expiration or for other reasons. The capture of these events will be done automatically by the system or by means of authorized entities and users.

Optionally, it also contemplates:
Obtaining information on a collection, donation, manufacture, utilization or clinical intervention process (date, time, type of product, origin, destination, . . . ), store this information in the corresponding vaults and profiles and, optionally, notify the owner or entity responsible for the process (right analytical result, for example).
Obtaining relevant information from other entities (medical centres, health authorities, . . . ) and incorporate it into the stored vaults and profiles, either for public access to said information or for private access from authorized entities and personnel.
Obtaining data from the product itself using telemetry devices incorporated in the product or devices to which the product is associated (such as geolocation, temperature and other variables), store them in the database and update product information when results are available.
Generate automatic alerts when certain conditions are met, such as variation in temperature, near expiration or product wastage for a specific cause (invalid product, expired date, . . . ).
Send a global alarm to all parties involved and related to the process after detecting a serious illness in a donor or a serious anomaly in any of the related processes (collection, production, supply, utilization) to initiate protocols and check donations or related products that may be affected.
Store in the product profiles and/or in the generated universal product code identifiers of one or more countries (GUDID, EU-UDI, . . . ) and one or more product labeling systems (ISBT 128, GS1, HIBC, . . . ).
Associate the product with one or more links to web addresses in the identification code of the product generated and/or in the public information of the product, so that any user can consult commercial information or public access data related to the product.

Going into more detail it should be noted that, the unified code for the identification of a biologic originated product generated according to the procedure of the invention will be UDI compatible by containing both UDI-DI and UDI-PI and will be deposited or printed on the product itself univocally and non-transferable, by means of a human readable alphanumeric code (HRI) and a machine readable code to be read automatically from an electronic device (MRI) and carried out preferably on a NFC tag without ruling out other options (such as a QR code, for example), and includes a series of data that identify and distinguish it as unique.

The identification code for the products and for the other related parties, in addition to containing the public identifier that allows access to public data stored in the system, may optionally contain the private identifier for access to private information (which can only be accessed and deciphered by an authorized device and personnel), and may also include information to be legible and consulted by any user such as production date, blood group, expiration, etc. Optionally, said identification code, once generated, can also be stored in the database of the computer system accessible by the different parties and/or users (producer or manufacturer, intermediaries, customers, recipients, consumers, . . . ).

According to the above, the procedure will allow generating, global public and private identifiers, unique and non-transferable, as well as the unique identification codes of:
products of biological origin;
individuals (human or other types) as donors, clients, users and patients;
cellular production lines;
entities such as companies, organizations, donation centres, medical centres, clinics, hospitals, laboratories, as well as related personnel and devices to access the system;
clinical trials, clinical interventions and treatments whether medical in humans or veterinary in animals.

It is remarkable that, on one hand, the identification code for an individual may contain certain health and medical information for emergencies that the user has approved for publicly sharing such as blood group, prescribed medication, allergies and contacts for emergencies. On the other hand, and as security measures, the system will notify the individuals and/or their contacts when the information of the identification code is read and the user and/or the device, entity and personnel that makes the reading will be identified. Furthermore, if access to private medical information is required by an entity and/or personnel to which the individual has not granted permission, the access permissions will be requested to the individual and/or to the contacts established for such case.

Going into more details, the public identifier will be composed of a set of alphanumeric characters called “error-free” that cannot be confused with each other due to wear, dirt or bad printing of them, in order to avoid errors in the identification. This set will consist of only 16 characters that form an alphanumeric-hexadecimal map translation and that is stored in the system, formed by but not limited to “ABEHKMNOSWXYZ349”. Said identifier will preferably have a length of 32 characters, so that the number of possible public identifiers using this format is 16 raised to 32, or about 3.4×10 raised to 38. Each of the characters will have an equivalence with a hexadecimal number using the defined translation map, and this equivalence may vary with each version of the public identifier that is implemented. On the other hand, said product identifier may contain a series of characters that allow determining the version of the translation map used and also one or more redundancy characters to perform the automatic error detection and correction by the system when the identifier is read.

The direct translation of alphanumeric character to hexadecimal number by means of the character translation map facilitates the storage of the public identifier in the system, which will consist of 32 hexadecimal digits. The public identifier may be stored in the system, in a non-limiting manner, in a variant of the UUID format (Universally Unique Identifier).

On the other hand, the creation of translation maps of alphanumeric characters to hexadecimal numbers adds an extra layer of security to avoid that the generation system of public identifiers can be discovered and makes it possible to change the alphanumeric coding of the public identifier simply by assigning a new map translation of alphanumeric characters, without the need to make other modifications in the identification system.

It should be noted that each of the data profiles associated with the global storage vaults are generated either by the authorized entity and personnel that performs the registration or creation of the global storage vault in the system or by any of the other authorized related parties in the system for data storage, through an electronic device (computer, tablet, etc.) and a software.

With all the above, thanks to the identification code generated, the identification of the product or any of the other related parties can be made by a computer equipment or electronic device conveniently installed for this purpose and, through the identification data of the code and the corresponding access permissions, to access to the data of the vaults and profiles stored in the system to which the access permission has been granted in each case.

In case of existence of any impediment to generate the registration of a data vault or profile in the system, access to specific data, make a transaction (purchase/sale, sending/receiving, . . . ) or for the utilization of a product (absence of analytical, breakage of the cold chain, incompatibility, . . . ), the computer system detects it thanks to the information contained and accessible through the unified code, being able to generate an alarm (visual, audible) that alerts of this circumstance to the related parties and to the corresponding personnel.

The generated identification code also allows the content of the information it incorporates to be varied and, consequently, the automatic updating of the details of the product data and the associated profiles in the database accessible by the different authorized parties (date storage or fractionation, delivery/collection, etc.).

Optionally, the computer system will be able to capture and associate to the data vaults and data profiles of each product results of laboratory analysis and other data of the production and/or supply chain, or data from telemetry devices incorporated in the product or from other devices to which the product is associated (such as geolocation and/or temperature) and store them in the database accessible by the different parties. The system may incorporate all or part of said additional information that will be introduced into the product data and the corresponding associated data profile, either automatically or selectively through a software or application with connection to the system.

In addition, the computer application may send a notification to the electronic device of an individual (donor, user, client, recipient, patient) and/or the entity (s) and designated and/or authorized personnel when there are updates on the status of any type of information with which it is related (analytical received, close expiration, etc.).

Optionally, the computer system may alert related parties and designated personnel to receive notices by receiving relevant information from authorized entities/centres or from telemetry devices (e.g. in case of detection of a relevant disease in a donor or breakage of the cold chain) and incorporate said information into the corresponding data vault and/or data profile.

The system can also automatically detect the location of the products and inform the related entities and designated personnel to receive this information by generating notifications, as well as indicate an estimated time of arrival of a product, for example for the performance of an organ transplant or a blood transfusion. The detection of the product will be made through geolocation means that incorporate the product itself such as those included in its packaging or through electronic devices to which the product is associated, such as a SIM card through a mobile phone or other geolocation device provided by the service and/or personnel carrying out the transport.

In case of detection by the system of any discordance or absence of certain data entered, stored, collected or received, the system may assess the situation and warn the related parties (collector, producer, transport, recipient, etc.), and mark the related data as erroneous or pending verification. For example, if the registration of a product is made from a universal donor identifier and the blood group associated with the donor does not coincide with the result of the blood test, the system will warn of this situation and mark the product as pending of verification and not suitable for use. Likewise, the rest of the bags of blood collected on the same day by the same donation centre or analyzed on the same day by the same laboratory can be marked automatically to request a verification of the rest of the products that may be involved.

The discreet procedure for the unified global registry and universal identification of products of biological origin for medicinal purposes in humans or other animals and of the different related parties consists of an object of unknown characteristics thus far for the purpose for which it is intended, reasons which, combined with its practical utility, provide it with sufficient grounds to obtain the privilege of its exclusivity which is required.

In view of the described FIGURE, and in accordance with the numeration adopted therein, it can be seen how the procedure essentially comprises of the following phases and steps:

In the first phase (A)
i. Generation of:
   a) a global and unique data vault for each of the parties, carried out after a voluntary request made by a public user or by one of the related authorized parties.
   b) one or several data vaults associated with the global vault containing data stored in each specific geographic region or territory;
   c) one or several data profiles associated with each vault for each part after the creation of the data vault in the system and that store public information of the product accessible to any user and private information that is managed and shared by the different related parties;
   d) a unique and non-transferable public identifier in hexadecimal format associated with the global data vault;
   e) a unique and non-transferable private identifier in hexadecimal format associated with the global data vault;
   f) one or several alphanumeric-hexadecimal translation maps associated with the public identifier and with the private identifier, formed by a set of error-free characters such as ABEHKMNOSWXYZ349 or similar;
   g) a unique public identification code containing at least one field for storing the public identifier in alphanumeric format, generated from the hexadecimal public identifier and a concrete alphanumeric-hexadecimal translation map, and optionally other fields for storing the private identifier and/or other data, to which the public identifier, the global data vault and the rest of the generated local vaults and data profiles are associated;
ii. Storage of the data of the generated vaults and profiles, the private identifier, the public identifier, the translation map and the identification code in a database accessible by the different parties according to their access rights to the data.
iii. Automatic updating by the system of vault and profile data with relevant events and the date on which they occurred (clinical interventions, diseases, donations, allergies, adverse reactions, . . . );
   In a second phase (B):
iv. Identification of the global data vault and the type of data stored (product, individual, clinical intervention, entity, . . . ) by the system executed by a public user or by an entity and its authorized personnel through the public identification code, for access to the information stored in the system contained in its data vaults and in the associated profiles based on the access permissions that are available when making the identification;
v. Generation, registration and automatic communication of an incident in case of existence of any impediment when making the identification, accessing the data, making the registration of new information, when a verification is required or when certain previously established conditions are met (variations in the temperature, close expiration, rejection of the product for some reason, . . . ).
vi. Automatic updating of the information in the vaults and profiles stored in the database accessible by the different parties with data on the process of obtaining, manufacturing, supplying and/or using (lot number, result of analysis performed, shipping/reception transactions, utilization in a clinical intervention, data of the individuals, . . . ).

In addition, optionally, the procedure contemplates, in the use phase:
vii. Automatically obtain information from a collection, donation, supply, utilization or clinical intervention process and store this information in the corresponding vaults and profiles;
viii. send a notification to the related and/or designated parties when the data collected is available for consultation.

Optionally, the procedure also contemplates the incorporation of one or several of the following stages:
ix. Obtain relevant information from other entities (collection of reports, tests and analytics, detected diseases, etc.) and automatically incorporate this information into the data and profiles of the vaults, and notify of it.
x. Generate automatic alerts when it is detected that certain conditions are met (close expiration, expiration of a product, detection of a disease or anomaly, . . . ), to initiate protocols, request verification or validation of other data or related products that may be affected and incorporate the information to the corresponding data vaults and profiles.
xi. Detect and register the location of a product through geolocation incorporated in it or through electronic devices to which it is associated (SIM card by mobile phone, geolocation device, . . . );
xii. Generate a notice and inform of the location of a product to the owner and/or to the destination, as well as the expected delivery or arrival time of the product;

Finally, if a new data profile is generated in the vaults when a transaction is made (sending/receiving, buying/selling, utilization in a clinical intervention, etc.), the procedure optionally contemplates:
xiii. Check that the conditions to carry out the transaction are met and notify the parties (valid or compatible product, authorized recipient to store data in the system, etc.);
xiv. update the information in the data vaults and the information in the profiles with the result of the transaction made.

In a preferred embodiment, the procedure generates global public and private identifiers, unique and non-transferable, as well as the unique identification codes of:
products of biological origin;
individuals (human or other types) as donors, clients, users and patients;
cellular production lines;
entities such as companies, organizations, donation centres, medical centres, clinics, hospitals, laboratories, as well as related personnel and devices;
clinical trials, clinical interventions and treatments, whether medical in humans or veterinary in animals.

In a preferred embodiment the identification code is deposited on an NFC tag with a unique identifier or similar;

In another preferred embodiment, the identification code is deposited in a QR code or similar;

In a preferred embodiment the identification code contains two types of information; a first type of public information other, private information, encrypted and only accessible by the authorized entities and personnel through a secure key known point-to-point between the device that obtains permission to access the encrypted data and the computer system that grants access to said data.

In a preferred embodiment, the identifier of the individuals (donors, recipients, customers, users, patients) is made by using the unified donor identifier as a means of universal identification and for the traceability of individuals in both directions (from origin to destination and from destination to origin).

In a preferred embodiment, the public identifier and the private identifier are formed by 32 hexadecimal characters.

In a preferred embodiment, the public identifier and the private identifier are generated in a variant of the UUID format.

In a preferred embodiment of the invention, the internal communication between the computer system of the invention and the different computer systems of each authorized entity/centre will be carried out on a secure common medium for the exchange of data and data storage, such as a common API, communication through VPN or blockchain technology and Smart contracts, being able to include one or several of these options and without excluding other options for this purpose.

As the attributes of the presented invention have been sufficiently described, as well as how to put it into practice, it is not considered necessary to make a more extensive explanation so that any expert in the matter understands its scope and the advantages that derive from it, clarifying that, within its essence, it may be put into practice in other ways of implementation that differ in detail from the application thereof indicated by way of example, and which will also reach the protection that is sought, provided that it is not altered, changed or modified in its fundamental principle.

What is claimed is:

1. A procedure for unified global registry and universal identification of products of biological origin for medicinal purposes in humans or other animals and of the different related parties, wherein the procedure comprising:
In a first phase (A):
i) Generation of:
a. a global and unique data vault for each of the parties, carried out after a voluntary request made by a public user or by one of the related authorized parties;
b. one or several data vaults associated with the global vault containing data stored in each specific geographic region or territory;
c. one or several data profiles associated with each vault for each part after the creation of the data vault in the system and that store public information of the product accessible to any user and private information that is managed and shared by the different related parties;
d. a unique and non-transferable public identifier in hexadecimal format associated with the global data vault;
e. a unique and non-transferable private identifier in hexadecimal format associated with the global data vault;
f. one or several alphanumeric-hexadecimal translation maps associated with the public identifier and with the private identifier, formed by a set of error-free characters such as ABEHKMNOSWXYZ349 or similar;
g. a unique public identification code containing at least one field for storing the public identifier in alphanumeric format, generated from the hexadecimal public identifier and a concrete alphanumeric-hexadecimal translation map, and optionally other fields for storing the private identifier and/or other data, to which the public identifier, the global data vault and the rest of the generated local vaults and data profiles are associated;
ii) Storage of the data of the generated vaults and profiles, the private identifier, the public identifier, the translation map and the identification code in a database accessible by the different parties according to their access rights to the data.
iii) Automatic update by the system of the data storage vault and/or profile data with relevant events and the date on which they occurred (clinical interventions, diseases, donations, allergies, adverse reactions, . . . );
In a second phase (B):
iv) Identification of the global data vault and the type of data stored by the system executed by a public user or by an entity and its authorized personnel through the public identification code, for access to the information stored in the system contained in its data vaults and in the associated data profiles based on the access permissions that are available when making the identification;
v) Generation, registration and automatic communication of an incident in case of existence of any impediment when making the identification, accessing to the data, making the registration of new information, when a verification is required or when certain previously established conditions are met (variations in the temperature, close expiration, rejection of the product for some reason, . . . ).
vi) Automatic updating of the information in the vaults and profiles stored in the database accessible by the different parties with data on the process of obtaining, manufacturing, supplying and/or using (lot number, result of analysis performed, shipping/reception transactions, utilization in a clinical intervention, data of the individuals, . . . ).

2. The procedure according to claim 1, wherein the procedure generates global public and private identifiers, unique and non-transferable, as well as the unique identification codes of products of biological origin, of individuals (human or other types), of cellular production lines, of entities (companies, organizations, donation centers, medical centers, clinics, hospitals, laboratories, . . . ) as well as of the related personnel and devices, and of clinical interventions (medical in humans or veterinary in animals).

3. The procedure according to claim 1, wherein the procedure uses the universal donor identifier as the universal identifier of individuals and for the traceability of the health history of all related individuals (donors, recipients, users, clients, patients).

4. The procedure according to claim 1, wherein the product identification code contains product identifiers from one or more countries, one or more product labeling systems and, additionally, one or more links to web addresses.

5. The procedure according to claim 1, wherein the identification code is deposited in a device that contains a unique identifier such as an NFC tag or similar.

6. The procedure according to claim 1, wherein the identification code is deposited and printed in QR code format or similar.

7. The procedure according to claim 1, wherein the public identifier and the private identifier are formed by 32 hexadecimal characters and, additionally, a variant of the UUID format is used.

8. The procedure according to claim 1, wherein the public identifier, the private identifier and, additionally, other data contained in the identification code are available for automatic reading by electronic devices (MRI) and for human reading (HRI) by means of a set of alphanumeric characters that avoid confusion among them by a bad reading and that are defined by an alphanumeric-hexadecimal translation map.

9. The procedure according to claim 1, wherein the identification code contains two types of information; a first type of information unencrypted and other information encrypted and only accessible by entities and authorized personnel through a secure key known point-to-point between the device that obtains permission to access the encrypted data and the computer system that grants access to said data.

10. The procedure according to claim 1, wherein the procedure also comprises:
vii) Automatically obtain, during the process of collection, manufacture, supplying and utilization, the information related to each of the parties (production data, derived products, telemetry devices, . . . ) and storing said information in the vault data and in the corresponding profiles;
viii) Send a notification to the related and/or designated parties when the obtained data can be consulted;
ix) Obtain relevant information from other entities (collection of reports, tests and analysis, detected diseases, etc.) and automatically incorporate this information into the data and product profiles, and notify it;
x) Generate automatic alerts upon detection of certain conditions (close expiration, expiration of a product, detection of a disease or of an anomaly, . . . ), to initiate protocols, request verification or validation of other data or related products that may be affected and incorporate said information into the vaults and the corresponding data profiles;

11. The procedure according to claim 1, wherein the procedure also comprises:
xi) Detect and register the location of a product through geolocation incorporated in it or through electronic devices to which it is associated (SIM card by mobile phone, geolocation device, . . . );
xii) generate a notice and report the location of a product to the owner and/or to the destination, as well as the expected delivery or arrival time of the product;

12. The procedure according to claim 1, wherein a new data and/or a new data profile are added in the data vaults when a transaction is made (sending/receiving, buying/selling, utilization in a clinical intervention, etc.), optionally the procedure comprises:
xiii) Check that the conditions to carry out the transaction are met and notify the parties (valid or compatible product, authorized recipient to store data in the system, etc.);
xiv) update the data of the data vaults and/or the information contained in the profiles with the result of the transaction made.

13. The procedure according to claim 1, wherein the internal communication between the computer system of the invention and the different computer systems of each authorized party will be performed on a common medium safe for the data exchange and data storage.
